(19) United States
(12) Patent Application Publication
(10) Pub. No.: US 2010/0262115 A1
(43) Pub. Date: Oct. 14, 2010

(54) NANOPARTICLES FOR CANCER SONODYNAMIC AND PHOTODYNAMIC THERAPY

(75) Inventors: Ragupathy MADIYALAKAN, Edmonton (CA); James XING, Edmonton (CA); Thomas WOO, Edmonton (CA); Eric SWANSON, Edmonton (CA)

Correspondence Address: EDWARD YOO C/O BENNETT JONES, 3200 TELUS HOUSE, SOUTH TOWER, 10020 - 100 STREET, EDMONTON, ALBERTA, AB, T5J 0N3, CA

(73) Assignees: INTELLIGENTNANO INC., Edmonton (CA); QUEST PHARMATECH INC., Edmonton (CA)

(21) Appl. No.: 12/755,942

(22) Filed: Apr. 7, 2010

Related U.S. Application Data
(60) Provisional application No. 61/167,403, filed on Apr. 7, 2009.

(51) Int. Cl. A61M 37/00; A61K 9/14; A61K 31/498; A61K 39/44; A61K 51/12; A61P 35/00

(52) U.S. Cl. 604/500; 424/497; 514/250; 424/178.1; 424/1.37; 977/915

(57) ABSTRACT

The present invention is directed to nanoparticles comprising a cancer therapeutic, pharmaceutical compositions comprising same, and methods for using same for drug delivery and ultrasound or light-based treatment of cancer.

### CROSS REFERENCE TO RELATED APPLICATION

This application claims the priority benefit of U.S. Provisional Application No. 61/167,403 filed on Apr. 7, 2009 entitled “Nanoparticles for Cancer Sonodynamic and Photodynamic Therapy,” the contents of which are incorporated herein by reference.

### FIELD OF THE INVENTION

This invention relates to nanoparticles comprising a cancer therapeutic agent, pharmaceutical compositions comprising same, and methods for using same for drug delivery and sonodynamic or photodynamic treatment of cancer.

### BACKGROUND OF THE INVENTION

Radiation therapy and chemotherapy are conventional treatments for cancer. Radiation therapy involves delivery of an optimal dose of either particulate or electromagnetic radiation to a particular area of the body with minimal damage to normal tissues. The source of radiation may be outside the body of the patient or may be an isotope implanted or instilled into the body. Chemotherapy involves treatment by chemical agents. However, both radiation and chemotherapy may harm healthy cells, resulting in undesirable systemic reactions including malaise, fatigue, loss of appetite, nausea, vomiting, headache, pain and hair loss.

Photodynamic therapy (PDT) involves a compound known as a photosensitizer which can be excited by visible or near infrared light of a specific wavelength. The compound is administered to a patient for delivery to the target tissue which is then illuminated, activating the photosensitizer to destroy the target tissue by generating singlet oxygen. Photodynamic therapy is mainly limited to superficial and/or small lesions since light cannot penetrate through more than about one centimetre of tissue.

Sonodynamic therapy (SDT) uses ultrasound which is non-invasive, and is capable of focusing on malignancies deeper within tissue than PDT. SDT involves activating preloaded, non-toxic compounds known as sonosensitizers using ultrasound (Umemura et al., 1996; Green et al., 2001; Tachibana et al., 2008). Such compounds may be specifically absorbed in tumor cells, and produce cytotoxic effects upon activation by ultrasound.

Ultrasound waves can generate cavitations, which can be defined as the sonomechanical effect of the sound waves on micro-environmental gases within fluid. Both oscillating bubbles (or stable cavitations) and collapsing bubbles (inertial cavitations) are capable of producing damage to cell membranes. Using lower intensities of ultrasound, even below the threshold for inertial cavitations, apoptosis can be induced (Tachibana et al., 2008). The finding confirms that free radicals generated by ultrasound play a secondary role in apoptosis induction because free radicals are only produced at intensities above the threshold for inertial cavitations.

Possible mechanisms of SDT include generation of singlet oxygen or sonosensitizer derived radicals which have cytotoxic effects, or the physical destabilization of the cell membrane (Miyoshi et al., 1997). The effects of various sonosensitizers (e.g., nitrogen mustard) have been investigated on mouse leukemia L1210 cells in combination with ultrasound irradiation (Kremkau et al., 1976). Low-intensity ultrasound with no temperature increase showed similar results (Harrison et al., 1991).

Hypocrellins A and B are perylenequinone pigments isolated from the parasitic fungus Hypocrella bambusae. Such pigments have been traditionally used as Chinese medicines to treat rheumatoid arthritis, gastric diseases, and skin diseases related to fungal infections. Hypocrellins also exhibit photodynamic properties and the ability to generate singlet oxygen (Estey et al., 1996; Song et al., 1999; Ali et al., 2001; Yang et al., 2001a, 2001b).

However, hypocrellins are strongly hydrophobic (i.e., not water-soluble), making them problematic for clinical applications. Attempts have been made to modify their molecular structure chemically to make them water-soluble, including forming a complex with metal ions or liposomes or micelles (Zhang et al., 2008; Wang et al., 2007; Zhou et al., 2005; Zhao et al., 2004). Hypocrellins modified in these manners exhibit low cellular uptake and poor biological compatibility; for example, the compound in Wang et al. (2007) exhibited high cytotoxicity after exposure to visible light.

Therefore, there is a need for solubilization vehicles for the administration of hypocrellins. Further, the use of hypocrellins with PDT or SDT may provide a safer, more comfortable, alternative treatment for cancer patients.

### SUMMARY OF THE INVENTION

The present invention is directed to nanoparticles comprising a cancer therapeutic, pharmaceutical compositions comprising same, and methods for using same for drug delivery and sonodynamic or photodynamic treatment of cancer.

In one aspect, the invention is directed to a nanoparticle comprising an inner volume comprising a hypocrellin B derivative and a polyvinylpyrollidone shell encapsulating the inner volume.

In one embodiment, the nanoparticle is bound to a detectable labelling agent. In one embodiment, the detectable labelling agent is selected from a fluorescent or other light-emitting marker, a radioactive tracer, or a contrast agent. In another embodiment, the nanoparticle may be bound to a selective targeting moiety, such as an antibody or an antibody fragment.

In one aspect, the invention is directed to a pharmaceutical composition for treating a tumor in a subject comprising a nanoparticle as described herein in combination with one or more pharmaceutically acceptable carriers.

In another aspect, the invention is directed to a method of treating a tumor in a subject comprising the steps of:
(a) administering a nanoparticle comprising an inner volume comprising a hypocrellin B derivative and a polyvinylpyrollidone shell encapsulating the inner volume to the subject or the tumor; and
(b) applying ultrasound or light, or both ultrasound and light, to the tumor;
wherein the nanoparticle is internalized within the tumor sufficient to achieve a cytotoxic effect upon exposure to the ultrasound or light. The application of ultrasound and light may be simultaneous, or sequential in either order.

In one embodiment, the tumor may be a tumor of the brain, lung, breast, pancreas, kidney, colon, rectum, ovary, cervix or prostate. In one embodiment, the ultrasound or light is applied after an incubation period following administration of the nanoparticle. In one embodiment, the incubation period following application of ultrasound is at least thirty minutes. In one embodiment, the incubation period following application of ultrasound is at least about two hours, for example, between about two hours and four hours. In one embodiment, the incubation period following application of light is at least about four hours.

In another aspect, the invention is directed to a method for delivering a hypocrellin to a tumor comprising the step of contacting the tumor with an effective amount of a nanoparticle comprising an inner volume comprising a hypocrellin B derivative and a polyvinylpyrollidone shell encapsulating the inner volume such that the nanoparticle is internalized within the tumor.

### BRIEF DESCRIPTION OF THE DRAWINGS

The invention will now be described by way of an exemplary embodiments with reference to the accompanying simplified, diagrammatic, not-to-scale drawings:

FIG. 1 is a diagrammatic representation of (A) the structure of a water-insoluble hypocrellin (SL052) alone and in dimethylsulfoxide (DMSO); (B) the structure of polyvinylpyrollidone (PVP) alone and in water; (C) the structure of one embodiment of a water-soluble hypocrellin B derivative nanoparticle (SL052 NP). FIG. 1D shows the structure of hypocrellin B. FIG. 1E shows the structure of another hypocrellin B derivative (SL017).

FIG. 2 shows (A) a TEM photo of SL052-NPs; (B) a graph showing the effect of PVP concentration on the size of SL052-NPs as measured by dynamic light scattering; (C) a graph showing that the absorbance at 657 nm wavelength is linearly correlated with SL052 concentration between 0.042 and 85.9 μg/mL (correlation coefficient=0.995); (D) a graph comparing the UV-vis spectra of SL052 alone and the SL052-NPS nanoparticles.

FIG. 3 shows (A) dynamic light scattering measurement of SL052-NPs which are 1047 nm in size; (B) dynamic light scattering measurement of SL052-NPs which are 173.9 nm in size.

FIG. 4 shows (A) a graph comparing the cytotoxicities of SL052 alone and SL052-NPs exposed to immediate ultrasound treatment, and ultrasound treatment after a two hour incubation; (B) a graph showing the stability of SL052-NPs under different temperatures and storage durations; (C) a graph showing the results of a half maximal inhibitory concentration (IC50) test for SL052 after two minutes of ultrasound; and (D) a graph showing the results of a IC50 test for SL052-NPs after two minutes of ultrasound.

FIG. 5 shows (A) a graph showing the results of an IC50 test for SL052-NPs after cells were treated with 462 nm light; (B) a graph showing the results of an IC50 test of SL052-NPs after cells were treated with 617 nm light; (C) a graph comparing the cytotoxicity of differently sized SL052-NPs, with (a) being 131 nm, (b) being 150 nm, and (c) being 247 nm; (D) a graph showing that both SL052 and SL052-NPs are cytotoxic when exposed to ultrasound or light; and (E) a graph showing that the uptake of drug entering cells increases as the concentration of SL052-NPs increases.

FIG. 6 shows the effect of photodynamic therapy (PDT) or sonodynamic therapy (SDT) with SL052-NPs for tumors in vivo, with each group having three or four mice for PDT and five mice for SDT: (A) PDT with SL052-NPs (2 and 6 mg/kg); (B) PDT with SL052-NPs (4 mg/kg) for mice with migrated tumor; and (C) ultrasound/SL052 treated mice with abdominal SP/2 ascities producing tumors.

### DETAILED DESCRIPTION OF PREFERRED EMBODIMENTS

When describing the present invention, all terms not defined herein have their common art-recognized meanings. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims. The invention will now be described having regard to the accompanying Figures.

As used herein, the term “nanoparticle” means a particle having at least one dimension which is less than about 250 nm, and preferably in the range of about 50 nm to about 200 nm.

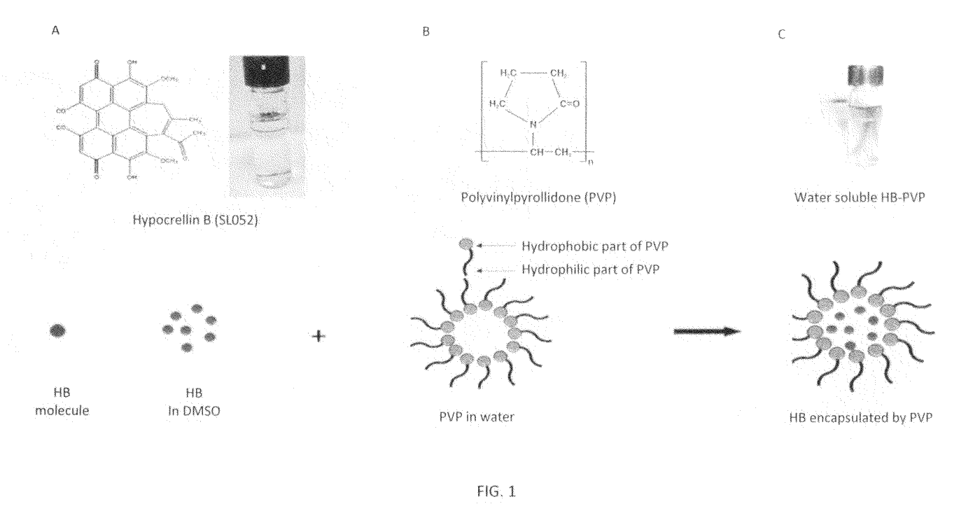

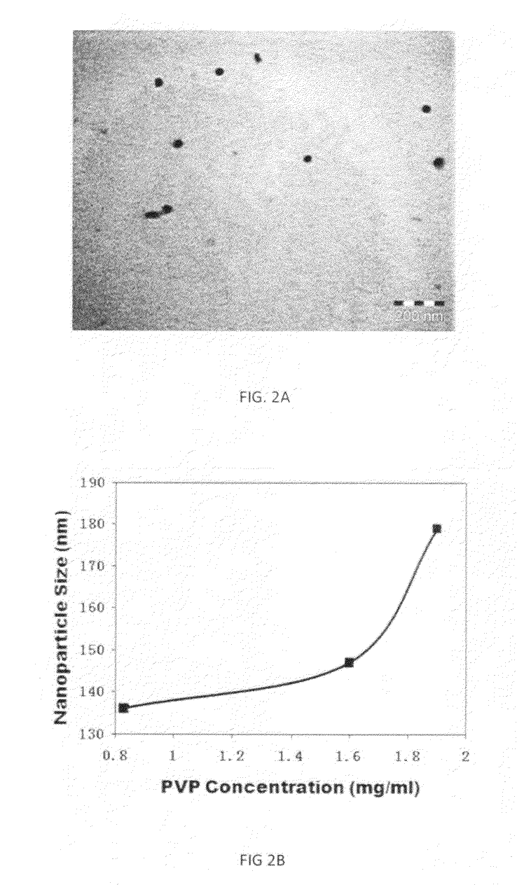

The present invention is directed to nanoparticles comprising a hypocrellin B derivative which is susceptible to photo- or sonodynamic activation. The nanoparticles may be used as a water-soluble drug for clinical applications including, but not limited to, intravenous injection or direct application to a tumor, for the treatment of cancers. A suitable hypocrellin for incorporation into the present invention may comprise a hypocrellin B derivative having anti-tumor activity. In one embodiment, the hypocrellin comprises an amino-substituted demethoxylated hypocrellin derivative having anti-tumor activity. In particular, suitable hypocrellins include those hypocrellin derivatives described in PCT International Publication No. WO 2007/016762 (Sharma et al.), the contents of which are incorporated herein, where permitted. In one preferred embodiment, the hypocrellin comprises the hypocrellin B derivative having the structure shown in FIG. 1A (designated as “SL052”). In another embodiment, the hypocrellin comprises the hypocrellin B derivative having the structure shown in FIG. 1E (designated as “SL017”).

It is well known that hypocrellins have relatively low absorption in the phototherapeutic window (600-900 nm) due to their longest wavelength absorbance band at 584 nm. To enhance absorbance in this visible red band, amino groups are introduced into hypocrellin molecules to enhance absorbance in this visible red band. One embodiment of a hypocrellin B derivative is a chemical derivative (SL052) of the parent hypocrellin β isolated from the parasitic fungus Hypocrella bambusae (Quest PharmaTech Inc., Edmonton, Canada). SL052 displays longer wavelength absorbance around 635 nm due to intra-molecular charge transfers between the amino and carbonyl group (Korbelik et al., 2009; Liu et al., 2008; Dickey et al., 2006; Xu et al., 2003). However, SL052 is strongly hydrophobic, tending to aggregate in aqueous solution. As shown in FIG. 1A, SL052 aggregates and floats on top of the water in the vial, rendering it problematic for clinical applications.

In one embodiment, nanoparticles of the present invention comprise an inner volume comprising a hypocrellin-B derivative and a polyvinylpyrollidone (PVP) shell encapsulating the inner volume. Such nanoparticles may be formed by self-assembly techniques. In one embodiment, nanoparticles comprising an inner volume comprising SL052 and a PVP shell may be prepared using a precipitation method, such as that described in Example 1 below. The hypocrellin-B derivative is dissolved in a suitable solvent which is miscible in water, such as DMSO, and is added to an aqueous solution of PVP. The resulting mixture may be stirred or agitated, and the nanoparticles spontaneously form in solution. In one embodiment, the PVP concentration in the final mixture may be varied from about 0.8 mg/ml to about 2.0 mg/ml, however, the precipitation method may be used at higher or lower concentrations of PVP.

In one embodiment, the hypocrellin-B derivative is first dissolved in DMSO in a concentration of about 3-6 mM, preferably about 4.6 mM. The dissolved SL052 is then added to the PVP solution in a volume ratio of about 1:2 to about 1:10. In one embodiment, the ratio is about 1:5.

PVP is a non-ionic, non-toxic water-soluble polymer (Kaneda et al., 2004) which has a hydrophilic portion and a hydrophobic portion. Compared with other polymers (for example, dextran, polyvinyl alcohol, polyacrylamide, polyethylene glycol and polydimethylacrylamide), PVP is a suitable polymeric carrier for prolonging the circulation of a hydrophobic drug, enhancing its permeability and retention, and localizing the conjugated drug in the blood. In one embodiment, PVP suitable for use with the present invention may have an average molecular weight of about 40,000.

Since the hydrophilic portion of PVP remains exposed in aqueous solution (FIG. 1B), the hydrophobic portion of PVP aggregates forms a hollow nanostructure shell to encapsulate SL052 as shown schematically in FIG. 1C. The SL052-PVP nanoparticles (SL052 NPs) are highly soluble and stable in aqueous solution, and avoid the problem of aggregation normally observed with SL052 alone. A large proportion of the SL052 molecules become encapsulated in the SL052 NPs.

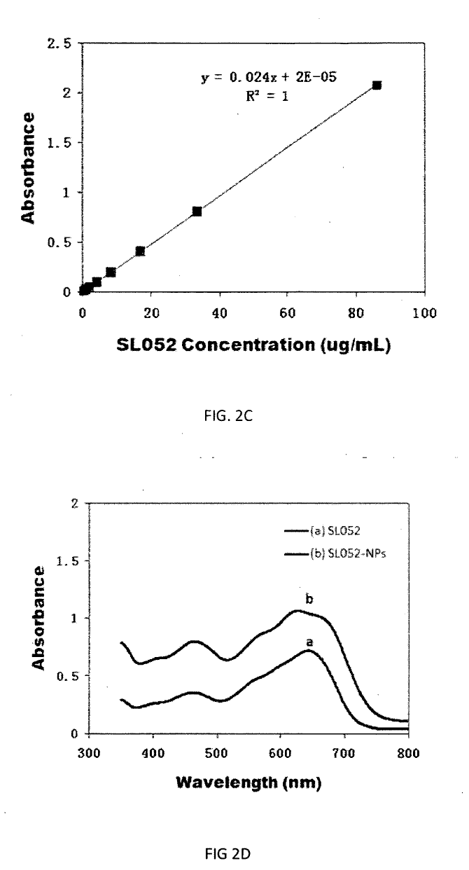

SL052-NPs were confirmed to be substantially spherical (Example 3, FIG. 2A) and substantially uniform in size. In one embodiment, the average size of the SL052-NPs is about 60 nm as determined by examining TEM photographs (FIG. 2A). Size determination by dynamic light scattering techniques provides results indicating a larger particle size, in the range of about 100-250 nm. Generally, the use of higher PVP concentrations results in larger nanoparticle size (FIG. 2B). The amount of SL052 which may be encapsulated can thus be estimated (Example 4).

The UV-vis absorption spectra of SL052 in DMSO and SL052-NPs in an aqueous solution are similar, with both groups exhibiting light absorption around 650 nm which locates in the red spectral region (FIG. 2D). These results indicate that SL052 retains its chemical structure and potential for PDT within the SL052-NPs.

In one aspect, the invention is directed to a pharmaceutical composition for treating a tumor in a subject comprising a nanoparticle described herein in combination with one or more pharmaceutically acceptable fluids or carriers. Those skilled in the art are familiar with any pharmaceutically acceptable carrier that would be useful in this regard, and therefore the procedure for making pharmaceutical compositions in accordance with the invention will not be discussed in detail. Suitably, the pharmaceutical compositions may be in the form of tablets, capsules, liquids, lozenges, lotions, aerosol, and solutions suitable for various routes of administration including, but not limited to, orally, via injection or infusion, intraperitoneally, topically, nasally, ocularly, vaginally or rectally, in solid, semi-solid or liquid dosage forms as appropriate and in unit dosage forms suitable for easy administration of fixed dosages.

As used herein, physiologically acceptable fluid refers to any fluid or additive suitable for combination with a composition containing the nanoparticles described. Typically these fluids are used as a diluent or carrier. Exemplary physiologically acceptable fluids include but are not limited to preservative solutions, saline solution, an isotonic (about 0.9%) saline solution, or about a 5% albumin solution or suspension. It is intended that the present invention is not to be limited by the type of physiologically acceptable fluid used. The composition may also include pharmaceutically acceptable carriers. Pharmaceutically accepted carriers include but are not limited to saline, sterile water, phosphate buffered saline, and the like. Other buffering agents, dispersing agents, and inert non-toxic substances suitable for delivery to a patient may be included in the compositions of the present invention. The compositions may be solutions, suspensions or any appropriate formulation suitable for administration, and are typically sterile and free of undesirable particulate matter. The compositions may be sterilized by conventional sterilization techniques.

Suitable methods and systems for sonodynamic therapy include, without limitation, those described in United States Patent Application Publication No. 2009/0062724 A1 to Chen, the contents of which are incorporated herein by reference, where permitted. Ultrasound may be applied to specific tissues using ultrasound transducers and methods similar to those used in ultrasound imaging methods.

Suitable methods and systems for photodynamic therapy include, without limitation, those described in WO/2008/011707 A1 to Woo et al, the contents of which are incorporated herein by reference, where permitted.

In one aspect, the present invention is directed to a method of treating a tumor in a subject comprising administering a nanoparticle to a tumor, and applying ultrasound or light to the tumor, or ultrasound and light to the tumor; wherein the nanoparticle achieves a cytotoxic effect to tumor cells upon exposure to ultrasound or light. The tumor may be a tumor of the brain, lung, breast, pancreas, kidney, colon, rectum, ovary, cervix or prostate.

As used herein, administering refers to any action that results in exposing or contacting the nanoparticles of the present invention with a pre-determined cell, cells, or tissue, typically mammalian. As used herein, administering may be conducted in vivo, in vitro, or ex vivo. For example, a composition may be administered by injection or through an endoscope. Administering also includes the direct application to cells of a composition according to the present invention. For example, during the course of surgery, tumor cells may be exposed. In accordance with an embodiment of the invention, these exposed cells (or tumors) may be exposed directly to a composition of the present invention, e.g., by washing or irrigating the surgical site and/or the cells.

In general, the nanoparticles may be injected intravenously which may result in general uptake by both healthy and tumor cells. The nanoparticles are then activated by localized light or ultrasound delivered to the tumour itself. Alternatively, the nanoparticles may be injected directly into the tumor, or otherwise applied directly to the tumor, to minimize uptake by healthy cells.

In one embodiment, the nanoparticles may be attached to a targeting moiety, such as an antibody or an antibody fragment which is specific for a tumor cell antigen. Thus, the nanoparticle may be delivered to a subject generally, and the nanoparticle will attach to the targeted tumor cells.

In one embodiment, ultrasound treatment or light treatment may be applied after an incubation period following injection or application of the nanoparticles, which incubation period may be 30 minutes or longer. In one embodiment, the incubation period is between about 2 hours to about 4 hours. Ultrasound treatment after a two hour or four hour incubation is generally more effective in killing cancer cells than immediate ultrasound treatment (FIG. 4A). Further, with an incubation period, SL052-NPs are more effective than SL052 alone in killing cancer cells when ultrasound is applied. Without restriction to a theory, it is believed that the SL052-NPs have an enhanced permeability through cell membranes, facilitating delivery and uptake of SL052 by cancer cells. In one aspect, the present invention is directed to a method for delivering a hypocrellin-B derivative to a tumor comprising contacting the tumor with an effective amount of a nanoparticle comprising an inner volume comprising a hypocrellin-B derivative and a polyvinylpyrollidone shell encapsulating the inner volume such that the nanoparticle is internalized within the tumor to release the hypocrellin-B derivative.

The SL052 NPs are stable for relatively long term storage. The stability of the SL052-NPs was determined by performing the same cytotoxicity test at different temperatures and storage durations (FIG. 4B). Freshly prepared SL052-NPs were mixed with water to yield a concentration of 50 μg/ml. Test samples were maintained at different temperatures (24° C., 4° C. and −20° C.). At different storage durations (one day, one week, one month, and two months), the efficacy of the test samples was tested using the MTT assay (described in Example 4). SL052-NPs remain effective for at least two months when stored at −20° C.

The cytotoxicities of SL052 and SL052-NPs were compared using the half maximal inhibitory concentration (IC50) test, with the IC50 value defined as the concentration of sonosensitizers needed to induce 50% cell killing. The IC50 values of SL052 and SL052-NPs are 60 μg/ml (FIG. 4C) and 24 μg/ml (FIG. 4D), respectively. The results indicate that SL052-NPs are more cytotoxic than SL052 alone or more sensitive to ultrasound activation than SL052 alone by a factor of 2.5.

SL052-NPs may serve as not only sononsensitizers, but also photosensitizers. Hypocrellins suitable for use with the present invention are known and effective photodynamic agents, and the SL052 NPs may be used in the same manner (see U.S. Pat. Nos. 6,627,664 and 7,157,477, the contents of which are incorporated herein by reference). Therefore, in one aspect, the invention comprises a method of treating a tumor in a subject comprising administering a nanoparticle comprising an inner volume comprising a hypocrellin-B derivative and a polyvinylpyrollidone shell encapsulating the inner volume to the subject; and applying light to the tumor; wherein the nanoparticle is capable of being internalized within the tumor sufficient to achieve a cytotoxic effect upon exposure to the light.

The examples provided herein indicate that SL052 NPs may be delivered to target cancer cells and generate cytotoxic radicals upon application of ultrasound or light. Without being bound by theory, ultrasound may act by striking the SL052-NPs and generating microcavities. Sonochemical effects, which directly relate to the high temperatures created during the collapse of microcavities, result in the disruption of chemical bonds and the formation of free radicals and other reactive ions. Such radicals cause DNA damage and induce apoptosis or necrosis of cancer cells. In one embodiment, ultrasound can penetrate into deep tissue or organs such as the prostate and pancreas. In another embodiment, light can be delivered in a more focussed manner than ultrasound. The SL052-NPs of the present invention enhance the effect of ultrasound by delivering SL052 to target cancer cells. The SL052-NPs display greater efficacy than SL052 alone, as evidenced by their ability to increase cytotoxicity upon application of ultrasound. Without being bound by theory, it is believed that the enhanced permeability of the SL052-NPs facilitates delivery and uptake of SL052 by cancer cells.

The nanoparticles of the present invention with fluorescent labelling can be used to study drug distribution in cells. When bound with PET or MRI tracers, SL052-NPs can be used for targeted delivery to trace cancer in vivo, and multiple imaging modality guided photo/sono therapy or thermotherapy for diagnostic and therapeutic purposes in the same, single platform. The detectable labelling agents to which the nanoparticles may be bound may be fluorescent or other light-emitting markers, radioactive tracers, or contrast agents as are well known in the art.

Therapy using SL052-NPs with either SDT or PDT, or both, is non-invasive with minimal, transient side-effects. Toxicity tests support the observation that the drug can be repeated as required, with no cumulated toxicity or “lifetime maximum dose.” Surgeons can selectively destroy cancers, while limiting the collateral destruction often caused by conventional surgery, cryotherapy and radiation. Unlike expensive radiation equipment, the ultrasound device to activate SL052-NPs is portable and may be useful to patients in developing countries or rural areas.

As will be apparent to those skilled in the art, various modifications, adaptations and variations of the foregoing specific disclosure can be made without departing from the scope of the invention claimed herein.

Exemplary embodiments of the present invention are described in the following Examples, which are set forth to aid in the understanding of the invention, and should not be construed to limit in any way the scope of the invention as defined in the claims which follow thereafter.

### Example 1

### Formation of SL052—PVP Nanoparticles

Polyvinylpyrollidone (PVP) (average molecular weight of 40,000) was purchased from Sigma Aldrich Canada Ltd. (Oakville, Canada), and an exemplary hypocrellin-B derivative (designated as “SL052”) was provided by Quest PharmaTech Inc. (Edmonton, Canada). The precipitation method was used to prepare SL052-NPS. Briefly, 1.5 mL of 0.5% (7.5 mg/ml) PVP aqueous solution was added to 6 mL of water with mixing at room temperature. After ten minutes, 1.59 mL of 4.6 mM SL052 in dimethylsulfoxide (DMSO, Fisher Scientific) was added to the mixture. The resulting solution was stirred for ten minutes under darkness to yield a nanodispersion with a nanoparticle size of 136 nm. The SL052-NPS were deep blue in color and water-soluble (FIG. 1C).

### Example 2

### Formation of Fluorescent SL052—PVP Nanoparticles

Fluorescent SL052 nanoparticles are formed by adding 1.5 mL of 0.5% PVP aqueous solution to 6 mL of water with mixing at room temperature. After ten minutes, 1.59 mL of 4.6 mM SL052 and 0.1 mM fluorescein isothiocyanate in DMSO was added to the mixture. The resulting solution was stirred for ten minutes under darkness to yield a nanodispersion.

SL052-NPs labelled with fluorescein isothiocyanate were used to treat cells for two hours with 6.25 mg/ml, 12.5 μg/ml, or 25 μg/ml SL052-NPs. Confocal microscopy confirmed that as the concentration of SL052-NPs increased, more SL052-NPs entered into cells, as may be seen in FIG. 2A.

### Example 3

### Determination of the Structures of the SL052-NPS

The structures of the SL052-NPS were determined by transmission electron microscopy (TEM). SL052-NPS were negatively stained with phosphotungstic acid and observed using TEM to confirm their spherical structure (FIG. 2A).

### Example 4

### Determination of the Sizes of the SL052-NPS

The size of the SL052-NPS can be adjusted by changing the PVP concentration (e.g., 7.5, 15.0 and 22.5 mg/ml), thereby enabling an estimation of the amount of SL052 which can be encapsulated (FIG. 2B). The volume of a single SL052 molecule is calculated as:

V_{molecule}=V_{C}+V_{H}+V_{O}+V_{N}=4π/3*(r_{C}³+r_{H}³+r_{O}³+r_{N}³)=17.2 [A³] [1]
wherein V_{molecule} is the volume of a single SL052 molecule;
V_{C}, V_{H}, V_{O} and V_{N} are the volumes of carbon, hydrogen, oxygen and nitrogen atoms in a single SL052 nanoparticle, respectively; and
r_{C}, r_{O}, r_{H}, r_{N} are the covalent radii of a carbon, a hydrogen, an oxygen and a nitrogen atom, respectively.

The volume of a single SL052-NP is calculated as:

V_{nanoparticle}=4π/3×(D_{nanoparticle}/2)³ [2]

wherein D_{nanoparticle} is the diameter of the SL052-NP as measured using TEM.
If D_{nanoparticle}=60 nm, V_{nanoparticle}=4π/3×(60/2)³×1000=1.1×10⁸ [A³]. Assuming that SL052 molecules are densely packed inside SL052-NPs, the number of SL052 molecules in a single SL052-NP is then equal to V_{nanoparticle}/V_{molecule} 1.1×10⁸/17.2=7×10⁶ molecules.

The number of SL052 molecules in a single SL052-NP per volume is calculated as:

(6.02×10²³×m/M)/(V×Number of SL052 molecules in a single SL052-NP)

(6.02×10²³×0.00048/592)/(7×10⁶)=3×10¹⁰ molecules/mL [3]

wherein m is the weight of SL052;

M is the molecular weight of SL052 (592); and

V is the solution volume (mL).

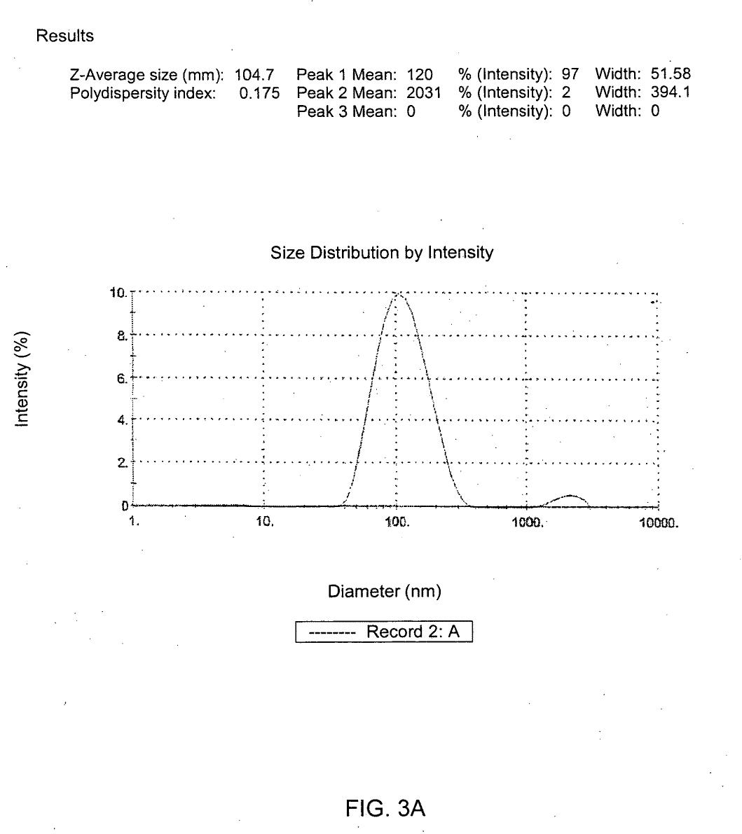

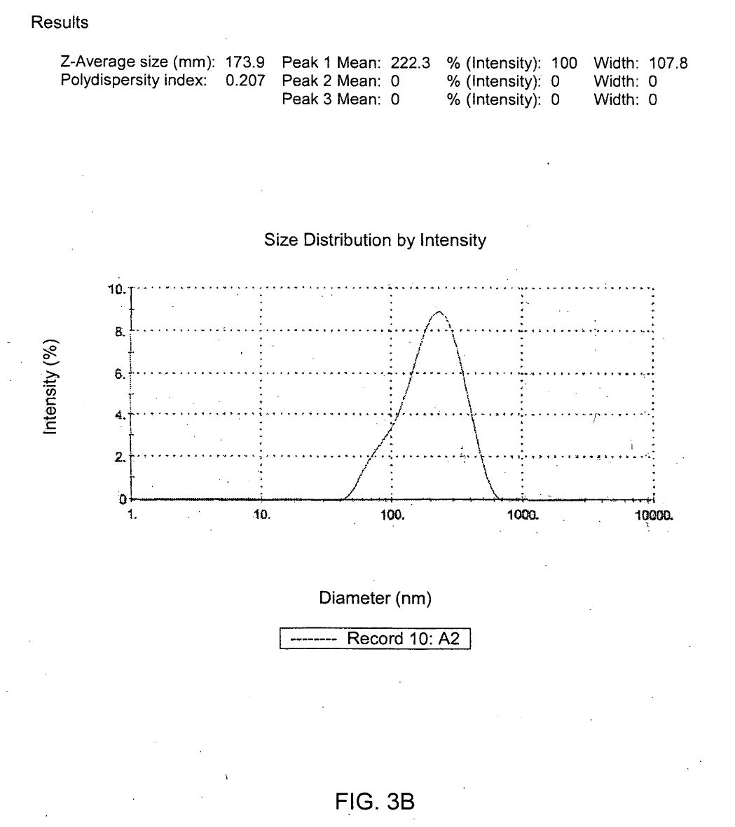

The sizes of the SL052-NPS were determined by dynamic light scattering using a Zetasizer™ Nano S (Malvern Instruments Ltd., Worcestershire, United Kingdom) (FIGS. 3A and 3B). UV-visible absorption spectra were obtained for size distribution by using a Lambda™ 900 UV/VIS/NIR spectrophotometer (PerkinElmer Life and Analytical Sciences, Woodbridge, Canada). The UV absorption values determined by the spectrophotometer with a wavelength absorbance at 657 nm were found to be linearly correlated with SL052 concentration within the range between 0.042 and 85.9 μg/mL (correlation coefficient of 0.995) (FIG. 2C). The SL052 concentration was 32.8 μg/mL. After dialysis for forty-eight hours, the SL052 concentration in nanodispersion was 27.7 μg/mL. These results indicate that 86% of SL052 was encapsulated within SL052-NPS, with few free SL052 molecules remaining in the solution.

### Example 5

### Determination of Ultrasound Intensity and Duration

Three test groups were prepared: (i) only Hela cells (control); (ii) Hela cells plus SL052 at a final concentration of 50 μg/ml; and (iii) Hela cells plus SL052-NPs at a final concentration of 80 μg/ml. The cells of each group were treated with ultrasound at different intensities (0.4 w/cm², 0.5 w/cm², 0.6 w/cm², 0.7 w/cm², and 0.8 w/cm²) for a duration of 1 minute, 2 minutes and 3 minutes, respectively. The medium was removed by washing the cells with phosphate buffered saline three times and 180 μl of fresh medium was added into each well. A MTT assay was then conducted to evaluate drug efficacy by adding 20 μl MTT (3-(4,5-dimethylthiazol-2-yl)-2,5-diphenyltetrazolium bromide) to each well, incubating for three hours and removing the medium. 200 μl of DMSO was added and the plate was lightly shaken for ten minutes to dissolve the insoluble purple formazan product into a colored solution. The absorbance of the colored solution was quantified by measuring at 490 nm using a spectrophotometer.

The MTT CellTiter 96 nonradioactive cell proliferation assay kit was purchased from Promega (Madison, Wis.). The ultrasound instrument used was a ULTRA SX made by EXCEL TECH LTD (http://www.xltek.com). The instrument can output ultrasound at 1 MHz with adjustable power intensities.

The testing result indicates minimal impact on Hela cells by applying one minute of ultrasound with ultrasound intensity at 0.6 w/cm². However, when the treatment time was extended to two or more minutes and the ultrasound intensity is greater than 0.6 w/cm², the ultrasound had obvious cell-killing effects (p<0.05). To avoid operational errors, the intensity of 0.7 w/cm² and 0.6 w/cm² with the duration of one minute and two minutes was chosen for SL052 and SL052-NP sonodynamic treatment experiments, respectively.

### Example 6

### Cell Cultures, Cell Uptake and Cytotoxicity Assay for Ultrasound and Light Activation

Human HeLa S3 cervical cancer cells (ATCC CCL-2) and SP/2 mouse myeloma cells (ATCC CRL 1581) were maintained in DMEM medium (Gibco Life Technologies) enriched with 10% heat-inactivated fetal bovine serum (FBS; Gibco) plus 100 UI penicillin G, and 100 μg/mL streptomycin (Sigma), and incubated under standardized conditions (37° C., 5% carbon dioxide, 100% humidity).

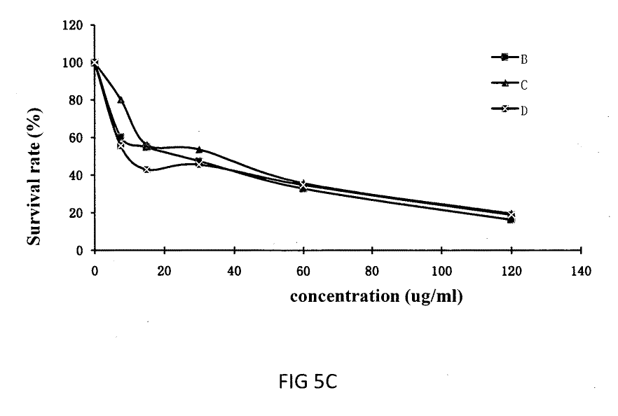

The concentrations of nanoparticle-encapsulated SL052 that were taken up by Hela cells in cell lysate were quantified using the UV absorption method. After the cells were exposed to various concentrations of SL052-NPs for two hours, the average SL052 drug value uptaken by each Hela cell was determined, as shown in FIG. 5E. After exposure to SL052-NPs at a concentration of 115 μg/ml for two hours, each cell uptook 75.23±4.38 pg SL052, which represents a marked increase when compared to the 2.46±0.32 pg uptaken by each cell when incubated with the same concentration of SL052 in water (not shown in FIG. 5E). Enhanced cell uptake of SL052-NPs was also directly confirmed by confocal microscopy (data not shown). The greater cell uptake of SL052-NPs compared to SL052 is likely due to the enhanced permeability of SL052-NPs. To achieve the same cell-killing effects, a reduced dosage of SL052-NPs may be used compared to a higher dosage of SL052 alone, reducing the collateral damage to normal tissue surrounding tumors.

Cell suspension (150 μl) was added into each well of a 96-well plate to provide 1×10⁴ Hela cells in each well. After incubating for twenty-four hours at 37° C., five test groups were prepared: (i) Hela cells (control); (ii) Hela cells plus SL052 at a final concentration of 50 μg/ml which receive immediate ultrasound treatment; (iii) Hela cells plus SL052-NPs at a final concentration of 50 μg/ml which receive immediate ultrasound treatment; (iv) Hela cells plus SL052 at a final concentration of 50 μg/ml which are treated with ultrasound after a two hour incubation; (v) cells plus SL052-NPs at a final concentration of 50 μg/ml which are treated with ultrasound after a two hour incubation. The cells were treated with ultrasound at an appropriate power and intensity. The medium was removed by washing the cells with phosphate buffered saline three times and 180 μl of fresh medium was added into each well. The MTT assay as described in Example 5 was then conducted.

Cell survival was determined by using a tetrazolium compound-based colorimetric method (MTT assay, Promega Celltiter 96) following the manufacturer's protocol. The data were recorded at the absorbance of 490 nm. The IC50 value at a given time point was calculated based on the concentration producing 50% reduction of absorbance value relative to solvent control absorbance value (100%) and expressed as mmol/L. The mean and standard deviation (SD) of IC50 were calculated based on the data obtained from three separate experiments, and six replicas were performed for each concentration dose.

The cytotoxicities of SL052 and SL052-NPs were compared using the half maximal inhibitory concentration (IC50) test, with the IC50 value defined as the concentration of sonosensitizers needed to induce 50% cell killing. The IC50 values of SL052 and SL052-NPs are 60 μg/ml (FIG. 4C) and 24 μg/ml (FIG. 4D), respectively. The results indicate that SL052-NPs are more cytotoxic than SL052 alone or more sensitive to ultrasound activation than SL052 alone by a factor of 2.5.

As shown in FIG. 2D, one absorption peak is around 462 nm and another peak is around 617 nm. A halogen light apparatus was set up, with the power and energy of the light being recorded. A 462 nm filter was then inserted in front of the light source, with the power and energy being again recorded. The halogen lamp was then turned on half an hour before measurement to provide a stable meter reading. Cells were treated with SL052-NPs and were then exposed under the filtered light for 30 seconds. Power (W) and energy (J) were recorded after each treatment. The IC50 value for cells treated with 462 nm light is 0.524 ml (FIG. 5A). Following the same procedure but replacing the 462 nm light filter with the 617 nm light filter, the IC50 value is 0.55 μg/ml (FIG. 5B). Both SL052 and SL052-NPs have cytotoxic effects when exposed to ultrasound or light (FIG. 5D).

### Example 7

### Sonodynamic Therapy Procedure

After the right dosage for the treatment was found, the cells were separated into five groups to determine the optimal sonodynamic therapy procedure: (i) the control; (ii) cells plus SL052 with a final concentration of 50 μg/ml which receive immediate ultrasound treatment; (iii) cells plus SL052-NPS with a final concentration of 50 μg/ml which receive immediate ultrasound treatment; (iv) cells plus SL052s with a final concentration of 50 μg/ml which receive ultrasound treatment after incubation; and (v) cells plus SL052-NPs with a final concentration of 50 μg/ml which receive ultrasound treatment after incubation.

The optimal time schedule for ultrasound treatment was investigated by exposing cells to SL052-NPS for different time periods and then administrating the ultrasound treatment. Ultrasound treatment after a two hour incubation was 20% more effective in killing cancer cells than immediate ultrasound treatment without incubation (p<0.05; FIG. 4A). It is believed that more SL052-NPS can enter cells following incubation and enhance the killing effects of ultrasound.

### Example 8

### Effect of Particle Size

The effect of nanoparticle size upon cytotoxicity was determined by testing SL052-NPs having different sizes, 131 nm, 150 nm and 247 nm (FIG. 5C). Serial dilutions of each sample of SL052-NPs were made (concentrations of 120 μg/ml, 60 μg/ml, 30 μg/ml, 15 μg/ml, and 7.5 μg/ml). The cells were treated with SL052-NPs at 37° C. for two hours and then ultrasound at an intensity of 0.56 w/cm² for two minutes. The IC50 results show that SL052-NPs, having a concentration between 9.5 μg/ml and 36 μg/ml and sizes between 131 nm and 247 nm, display similar cytotoxic effects upon application of ultrasound. Line A in FIG. 5C shows 131 nm results, line B shows 150 nm results, while line C shows 247 nm results.

### Example 9

### In Vivo Photodynamic Therapy Using a Murine Model

Male Balb/c mice were obtained from Charles River Laboratories International, Inc. (Wilmington, Mass.) and allowed to acclimatize for two weeks prior to testing. All mice had a bilateral flank implant of 1×10⁶ EMT-6 murine mammary tumor on each side. The tumors were allowed to grow to at least 5 mm in diameter before drug and light treatment. All treated mice received an intravenous tail vein injection of either 2 mg/kg, 4 mg/kg or 6 mg/kg nanoparticle solution. The injection contained 40 μL of the nanoparticle solution plus 60 μL of sterile saline to yield a 100 μl total volume injection. Two groups of nanoparticle formulation injected animals were used. The first group was treated with light four hours after injection. The second group was treated with light twenty-four hours after injection. The light treatment for both groups was 100 J/cm² at a wavelength of 650 nm with a fluency rate of 200 mW from a HPD diode laser via a 400 nm fiber. The tumor was measured and the longest axis was used to calculate the light spot size. The animals were anaesthetized and then draped during the light treatment except for the tumor area to prevent exposure in case of uptake by normal tissues. Once treatment was completed, the animals were left to recover and returned to their cages. The tumor response was monitored daily by caliper measurements of the tumor length, width and thickness. The following formula was used to calculate the tumor volume:

length×width×thickness×π/6 [4]

All tumor-bearing mice, which were treated with SL052-NPs at doses of 2 or 6 mg/kg and exposed to light, became tumor-free after light treatment after a 4 hour incubation period, except for one mouse which had been treated with a 6 mg/kg dose of SL052-NPs and light. The mouse was ill on day one post-treatment and died on day two. The cause of death was inconclusive from a gross postmortem examination. Mice treated with light 24 hours after injection of SL052 NPs remained healthy, but none of the animals had tumor ablation and had to be euthanized due to increasing tumor burden.

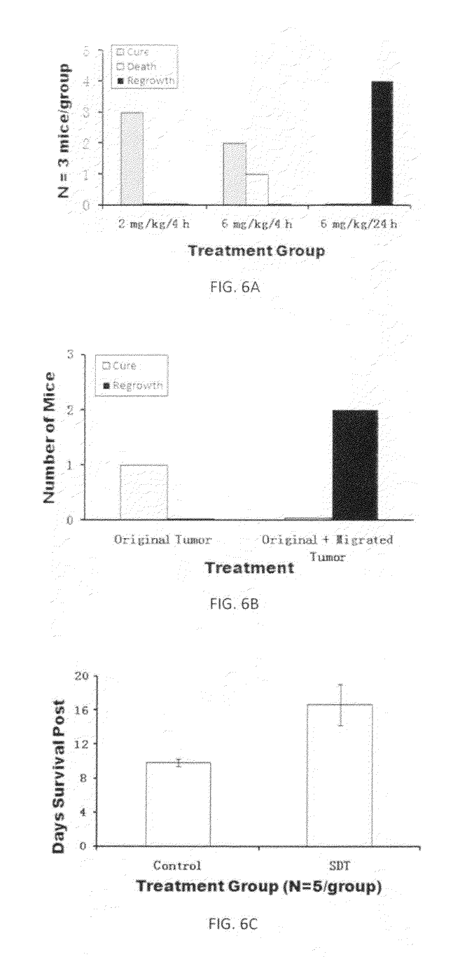

In a further experiment, mice bearing the original tumor were treated with a 4 mg/kg dose of SL052-NPs, with PDT after a 4 hour incubation period (FIG. 6B). After two weeks, the treated mice became tumor-free. In mice bearing both original and migrated tumors, the PDT was administrated at four hours post-treatment with SL052-NPs only to the original tumor. Without being bound to theory, re-growth of tumors in these mice may have been due to tumor-implanted cells which migrated away from the treatment site and were subsequently not PDT-treated, resulting in delayed but steady tumor re-growth.

### Example 10

### In Vivo Sonodynamic Therapy Using a Murine Model

Male Balb/c mice were given a priming dose of 400 μL of pristane (Sigma Aldrich Canada Ltd.). Thirteen days later, they were injected intraperitoneally with 5×10⁶ SP/2 mouse myeloma cells. Five control mice were left untreated after tumor implant. Five days post-tumor implant, the sonodynamic therapy treatment group was given an intra-peritoneal injection of 50 mg/kg SL052 NPs in Hank's Balanced Salt Solution (Sigma Aldrich Canada Ltd., total volume=0.75 mL). The mice were allowed a four hour drug uptake time in subdued lighting. They were then anaesthetized with sodium pentobarbital and subjected to ultrasound treatment at 1 MHz using a 50 mW power level for five cycles of two minutes of ultrasound followed by one minute without ultrasound to give a total delivered dose of 1.5 kilojoules. The animals were then left to recover and kept in subdued lighting for a further twenty-four hours to allow for drug metabolism/excretion.

The mice were then monitored daily for general health and body weight. The experiment ceased when animals became visibly distressed or death occurred overnight. For tumor-bearing mice, the survival days amounted to about seventeen days for SDT-treated mice compared with nine days for the untreated control (FIG. 6C).

### Example 11

### Statistical Analyses

Experimental values were determined in six replicas. All values regarding measurement were expressed as means and standard deviation (SD). The one-way analysis of variance (ANOVA) and Tukey multiple comparison post-test were used. Differences less than 0.05 (p<0.05) were considered statistically significant.

### REFERENCES

The following references are incorporated herein by reference (where permitted) as if reproduced in their entirety. All references are indicative of the level of skill of those skilled in the art to which this invention pertains.
Ali, S. M., Chee, S. K., Yuen, G. Y. and Olivo, M. (2001) Hypericin and hypocrellin induced apoptosis in human mucosal carcinoma cells. J. Photochemistry and Photobiology B: Biology 65:59-73.
Chen, R. System and apparatus for sonodynamic therapy. United States Patent Application Publication No. 2009/0062724 A1, published Mar. 5, 2009.
Dickey, D., Liu, W., Naicker, S., Woo, T., Moore, R. and Tulip, J. (2006) Using fluorescence to augment the efficacy of photodynamic therapy. Proc. SPIE 6343:634305.
Estey, E. P., Brown, K., Diwu, Z., Liu, J., Lown, J. W., Miller, G. G., Moore, R. B., Tulip, J. and McPhee, M. S. (1996) Hypocrellins as photosensitizers for photodynamic therapy: a screening evaluation and pharmacokinetic study. Cancer Chemotherapy and Pharmacology 37(4):343-50.
Frankamp, B. L. (Jan. 1, 2005) Directed Self-Assembly of Nanoparticles. Electronic Doctoral Dissertations for UMass Amherst. Paper AAI3193902.
Gelperina, S., Kisich, K., Iseman, M. D. and Heifets, L. (2005) The potential advantage of nanoparticle drug delivery system in chemotherapy of tuberculosis. American Journal of Respiratory and Critical Care Medicine 172:1487-1490.
Green, J., Kirway, J. J., Tierney, J., Symonds, P., Fresco, L., Collingwood, M. and Williams, C. (2001) Survival and recurrence after concomitant chemotherapy and radiotherapy for cancer of the uterine cervix: a systematic review and meta-analysis. The Lancet 358:781-786.
Harrison, G. and Balcer-Kubiczek, E. (1998) Continuous-wave ultrasound and neoplastic transformation in vitro. Ultrasound Med. Biol. 15(4):335.340.
Harrison, G. H. and Balcer-Kubiczek, E. K. (1991) Pulsed ultrasound and neoplastic transformation in vitro. Ultrasound Med. Biol. 17(6):627-632.
Kaneda, Y., Tsutsumi, Y., Yoshioka, Y., Kamada, H., Yamamoto, Y., Kodaira, H., Tsunoda, S., Okamoto, T., Mukai, Y., Shibata, H., Nakagawa, S, and T. Mayumi, T. (2004) The use of PVP as a polymeric carrier to improve the plasma half-life of drugs. Biomaterials 25(16): 3259-3266.
Korbelik, M., Merchant, S, and Huang, N. (2009) Exploitation of immune response-eliciting properties of hypocrellin photosensitizer SL052-based photodynamic therapy for eradication of malignant tumors. Photochemistry and Photobiology 85:1418-1424.
Kremkau, F. W., Kaufmann, J. S., Walker, M. M., Burch, P. G. and C. L. Spurr, C. L. (1976) Ultrasonic enhancement of nitrogen mustard cytotoxicity in mouse leukemia. Cancer 37 (4):1643-1647.
Liu, W., Dickey, D., Xiao, Z., Moore, R. and Tulip, J. (2008) Dosimetric considerations of interstitial photodynamic therapy of the canine prostate mediated by intra-arterially administered hypocrellin derivative. Proc. SPIE 6845:684519.
Miller, G. G. and Lown, J. W. Perylenequinones for use as sonosensitizers. U.S. Pat. No. 6,627,664, issued Sep. 30, 2003.
Miyoshi, N., Misik, V. and Riesz, P. (1997) Sonodynamic toxicity of gallium-porphyrin analogue ATX-70 in human leukemia cells. Radiation Research 148 (1):43-47.
Orive, G., Hernandez, R. M., Gascon, A. R. and Pedraz, J. L. (2005) Micro and nano drug delivery systems. Cancer Therapy 3:131-138.
Sanguansri, P. and Augustin, M. A. (2006) Nanoscale materials development—a food industry perspective. Trends in Food Science & Technology 17: 547-556.
Sharma, S. K., Woo, T. and Naicker, S. Perylenequinone Derivatives and Uses Thereof. International Publication No. WO 2007/016762, published Feb. 15, 2007.
Song, Y., An, J. and Jiang, L. (1999) ESR studies of the photodynamic properties of a long-wavelength and water-soluble hypocrellin B derivative: photogeneration of semiquinone radical anion and activated oxygen. Journal of Photochemistry and Photobiology A: Chemistry. 123(1-3):39-46.
Tachibana, K., Feril Jr., L. B. and Ikeda-Dantsuji, Y. (2008) Sonodynamic therapy. Ultrasonics 48:253-259.
Umemura, S., Kawabata, K., Sasaki, K., Yumita, N., Umemura, K. and Nishigaki, R. (1996) Recent advances in sonodynamic approach to cancer therapy. Ultrasonics Sonochemistry 3: S187-S191.
Wang, K., He, Q., Yan, X., Cui, Y., Qi, W., Duan, L. and Li, J. (2007) Encapsulated photosensitive drugs by biodegradable microcapsules to incapacitate cancer cells. Mater. Chem. 17:4018-4021.
Xu, S., Chen, S., Zhang, M. and Tao, S. (2003) Synthesis, characterization and photodynamic activity of phenmethylamino-demethoxy-hypocrellin B. Journal of Photochemistry and Photobiology B: Biology 72(1-3):61-67.
Yang, H., Zhang, W., Ma, L., Wang, S, and Zhang, Z. (2001a) An approach to enhancing the phototoxicity of a novel hypocrellin congener to MGC803 cells. Dyes and Pigments 51:103-110.
Yang, H., Wu, T., Zhang, M. and Zhang, Z. (2001b) A novel photosensitizer, 2-butylamino-2-demethoxy-hypocrellin B (2-BA-2-DMSL052)—its photodynamic effects on HeLa cells: efficacy and apoptosis. Biochemica et Biophysica Acta 1540(1): 22-31.
Zhang, M., Xu, S., Wu, T., Chen, S, and Shen, T. Aliphatic amino-substituted demethoxylated hypocrellins and their synthesis. U.S. Pat. No. 7,157,477, issued Jan. 2, 2007.
Zhang, H., Wang, D., Butler, R., Campbell, N., Long, J., Tan, B., Duncalf, D., Foster, A., Hopkinson, A., Taylor, D., Angus, D., Cooper, A. I. and Rannard, S. P. (2008) Formation and enhanced biocidal activity of water-dispersable organic nanoparticles. Nature Nanotechnology 3:506-511.
Zhao, B., Xie, J. and Zhao, J. (2004) A novel water-soluble nanoparticles of hypocrellin B and their interaction with a model protein: C-phycocyanin. Biochemica et Biophysica Acta 1670(2): 113-120.
Zhou, J., Liu, J., Xia, S., Wang, X. and Zhang, B. (2005) Effect of chelation to lanthanum ions on the photodynamic properties of hypocrellin A. J. Phys. Chem. B 109:19529-19535.

1. A nanoparticle comprising an inner volume comprising a hypocrellin-B derivative and a polyvinylpyrollidone shell encapsulating the inner volume.

2. The nanoparticle of claim 1, wherein the hypocrellin-B derivative is SL052.

3. The nanoparticle of claim 1, bound to a detectable labelling agent.

4. The nanoparticle of claim 3, wherein the detectable labelling agent is selected from a fluorescent or other light-emitting marker, a radioactive tracer, or a contrast agent.

5. The nanoparticle of claim 1, bound to a selective targeting moiety such as an antibody or an antibody fragment.

6. The nanoparticle of claim 1 in combination with one or more pharmaceutically acceptable carriers to form a pharmaceutical composition for treating tumors.

7. A method of treating a tumor in a subject comprising:
administering a nanoparticle claimed in claim 1; and
applying ultrasound or light to the tumor, or ultrasound and light to the tumor;
wherein the nanoparticle achieves a cytotoxic effect to tumor cells upon exposure to the ultrasound or light.

8. The method of claim 7, wherein the tumor is selected from a tumor of the brain, lung, breast, pancreas, kidney, colon, rectum, ovary, cervix or prostate.

9. The method of claim 7, wherein the ultrasound or light is applied after an incubation period following administration of the nanoparticle.

10. The method of claim 9, wherein the incubation period following application of ultrasound is at least thirty minutes.

11. The method of claim 9, wherein the incubation period following application of ultrasound is at least two hours.

12. The method of claim 9, wherein the incubation period following application of light is at least four hours.

13. The method of claim 7, wherein the nanoparticle is bound to a detectable labelling agent.

14. The method of claim 13, wherein the detectable labelling agent is selected from a fluorescent or other light-emitting marker, a radioactive tracer, or a contrast agent.
